(19) United States
(12) Patent Application Publication
(10) Pub. No.: US 2016/0012530 A1
(43) Pub. Date: Jan. 14, 2016

(54) Method and system to provide investment traders biofeedback in Real-Time during the decision time immediately prior to make or not make a transaction

(71) Applicant: Richard Gardner, Scottsdale, AZ (US)

(72) Inventor: Richard Gardner, Scottsdale, AZ (US)

(21) Appl. No.: 14/325,738

(22) Filed: Jul. 8, 2014

(51) Int. Cl. G06Q 40/04; A61B 5/0476; A61B 5/16; G06F 19/00; A61B 5/04

(52) U.S. Cl. CPC G06Q 40/04; G06F 19/345; A61B 5/0476; A61B 5/04012; A61B 5/16

(57) ABSTRACT

The present invention relates to system and method that analyze EEG brain wave data in real time throughout the day and correlating the data with the attention level of a person transmitting the EEG signals while he/she is working at a job. More particularly, the present invention is designed for investment day traders to evaluate their level of mental alertness and more particularly to correlate their level of mental alertness to their trading patterns but it is not limited to this industry sector.

### BACKGROUND OF THE INVENTION

1. Field of the Invention

The present invention relates to system and method that analyze electroencephalograph (EEG) brain wave data in real time throughout the day and correlating the data with the attention level of a person transmitting the EEG signals while he/she is working at a job. More particularly, the present invention is aimed for use by investment day traders to evaluate their level of mental alertness and more particularly to correlate their level of mental alertness to their trading patterns but it is not limited to this industry sector.

2. Description of Related Art

Biofeedback is well known in the art as an experimental technique but is not used or marketed for personnel in the work environment. EEG brain wave data is known as a good indicator of a person's attention level or alertness level and can be used as a useful biofeedback data for personnel in the work environment to assess and improve work performance.

Frequently a person's attention or alertness level is decreased under high stress at work. Therefore, it is desirable to apply EEG data to the industry sectors that are known to have high stress and challenges. Investment day traders are facing mental challenges every day they work. Day trading can be both mentally challenging and exhausting for the traders. Many losing trades occur when traders become fatigued or distracted. Profitable trades often occur when traders are in an optimal state of mental alertness, referred to as being “in the zone”. States of alertness and fatigue are usually perceived as a gray area. Traders frequently have difficulty entering into or knowing the optimal state of mental alertness associated with profitable trading.

Therefore, it is desirable to have a method and system that can apply EEG brain wave data in real time throughout the WORKING day and correlating the data with the attention level of a person transmitting the EEG signals while he/she is working at a job. More particularly it is desirable to have a method and system that can correlate their level of mental alertness to their trading patterns.

### SUMMARY OF THE INVENTION

The present invention relates to system and method that analyze EEG brain wave data in real time throughout the day and correlating the data with the attention level of a person transmitting the EEG signals while he/she is working at a job. More particularly, the present invention can be used by traders to evaluate their level of mental alertness and more particularly to correlate their level of mental alertness to their trading patterns but it is not limited to this industry sector.

Day traders have a high stress job. If a day trader could evaluate and analyze when he is under stress it would likely increase his probability of making better trades or deals. In an exemplary embodiment of the present invention, there is disclosed an invention which can help traders identify their mental state as being either alert or fatigued and provides biofeedback training based on trading patterns. The software analyzes the trader's brainwave patterns to provide discretionary alerts that inform when to either trade or not trade. The invention provides biofeedback that teaches the trader how to enter into an optimal state of alertness and how to maintain that alertness level.

The key elements of the present invention are (i) an EEG headset DEVICE, (ii) a desktop, laptop or tablet computer and (iii) software that analyze and correlate both trading data (i.e., success or failures) and EEG brain wave data. The EEG headset transmits raw EEG data to a desktop, laptop or tablet computer and perhaps a mobile phone. The software processes the EEG data, programmatically learning patterns that are EEG associated or correlated with winning or losing trades. The software is designed to alert the trader to either trade or not trade in real-time, based on the analysis of the trader's mental alertness which is based on the real-time EEG data analyzed by software specific for the purpose of linking mental alertness to successful trading.

An embodiment of the present invention includes an EEG headset worn by the trader to detect brain waves. The trader's raw EEG brainwave data is transmitted from the headset to the software that is loaded on a computer. The software allows the trader to analyze and trade the market as normal while wearing the EEG headset. The software analyzes the trader's profitability in association with his or her brainwave patterns. If the software is successful at correlating brainwave patterns with profitable and unprofitable trading outcomes, THESE correlations can be applied in real time to provide discretionary alerts to the trader when he/she is trading in a live market environment.

It is recognized that the present invention can be used in any work environment and is not limited to only personnel involved with day trading stocks, commodities or other items.

The more important features of the invention have thus been outlined in order that the more detailed description that follows may be better understood and in order that the present contribution to the art may better be appreciated. Additional features of the invention will be described hereinafter and will form the subject matter of the claims that follow.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

The foregoing has outlined, rather broadly, the preferred feature of the present invention so that those skilled in the art may better understand the detailed description of the invention that follows. Additional features of the invention will be described hereinafter that form the subject of the claims of the invention. Those skilled in the art should appreciate that they can readily use the disclosed conception and specific embodiment as a basis for designing or modifying other structures for carrying out the same purposes of the present invention and that such other structures do not depart from the spirit and scope of the invention in its broadest form.

### BRIEF DESCRIPTION OF THE DRAWINGS

Other aspects, features, and advantages of the present invention will become more fully apparent from the following detailed description, the appended claim, and the accompanying drawings in which similar elements are given similar reference numerals.

FIG. 1 is a diagram of an exemplary system for providing biofeedback to help making decisions on daily trades in accordance with the principles of the present invention.

FIG. 2 is a flowchart of an exemplary process for the method of providing biofeedback to help make decisions on daily trades in accordance with the principles of the present invention.

### DESCRIPTION OF THE PREFERRED EMBODIMENT

Day traders, at work, are under considerable pressure that results in stress to the individual. Stress can influence one's alertness and decisions in any discipline including day traders of stocks and other commodities. On the job, people can frequently recognize personal stress but this is a qualitative indicator. Thus, there is a need for a quantitative assessment of attention (or alertness), particularly in work environments that can cause stress to the employees.

Biofeedback is well known in the art as an experimental technique but is not used or marketed for personnel in the work environment. The present invention utilizes biofeedback to help individuals, on the job, to monitor their stress levels.

The present invention is a method to monitor attention (or alertness) in individuals attired with an EEG apparatus. An important element of the present invention is software that correlates attention (or alertness) levels by monitoring brain waves and using software to correlate the attention (or alertness) levels with performance on the job.

The present invention is aimed at quantitative assessment of EEG data as the data relates to a person's attention (or alertness). The primary element of the invention is software that correlates brain waves (monitored by the EEG device) with attention (or alertness) levels of personnel involved with trading stocks or other commodities.

Another element of the invention is any computer device capable of executing the software to display the personnel attention-information (or alertness-information) to the user who is equipped with an EEG receiver of brain waves.

Another element of the present invention is software that can display in real-time the attention (or alertness) level of the person equipped with an EEG receiver for brain waves.

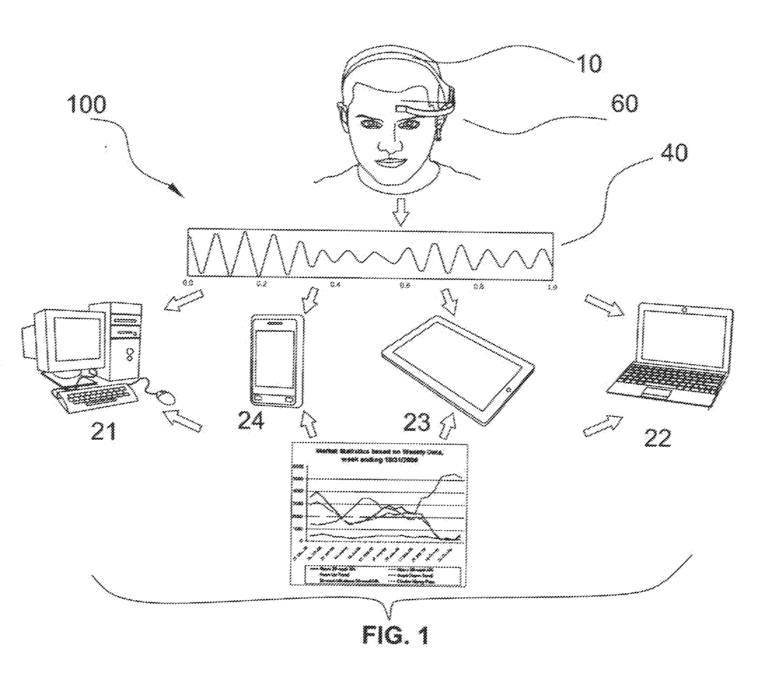

Referring to FIG. 1, there is disclosed the system in accordance with the principles of the present invention. The key elements of the invention 100 are (i) an EEG headset 10, (ii) a computer 20 including desktop 21, laptop 22 and tablet 23, or a mobile phone 24 and (iii) software (not visible) that analyzing and correlates both EEG brain wave data 40 and trading data 50 (i.e., success or failures). The EEG headset 10 transmits raw EEG data 40 to a desktop 21, laptop 22 or tablet computer 23 and perhaps a mobile phone 24. The software provides features for both trading and analyzing EEG data. The trading feature includes functionalities of typical trading software and is known in the field and thus is not discussed herein. The software processes the EEG data, programmatically learning patterns that are EEG associated or correlated with winning or losing trades. The software is designed to alert the trader 60 to either trade or not trade in real-time, based on the analysis of the trader's mental alertness which is based on the real-time EEG data analyzed by software specific for the purpose of linking mental alertness to successful trading.

An EEG headset 10 is worn by the trader 60. The trader's raw brainwave data 40 is transmitted from the headset 10 to the software that is loaded on the computer 20 or a mobile phone 24. The software 30 allows the trader to analyze and trade the market as normal while wearing the EEG headset 10. The software also analyzes the trader's profitability in association with his or her brainwave patterns. If the software is successful at correlating certain brainwave patterns with profitable or unprofitable trading outcomes, those correlations are applied in real time to provide discretionary alerts to the trader when trading with live market data.

In the past, EEGs equipment had to be placed on the scalp using gel and electrodes arranged in a plastic hat or cap. This invention can use a standard EEG cap, or it can use a more modern and portable EEG headset which requires no gel or medical knowledge for proper placement. The EEG device or data acquisition is irrelevant to the invention. Data is transmitted from whatever EEG capture device is being used, to the desktop, laptop or tablet computer using either a wired, wireless or Bluetooth data connection. Once the data has been transmitted to the computer or mobile phone the software will capture and analyze the data.

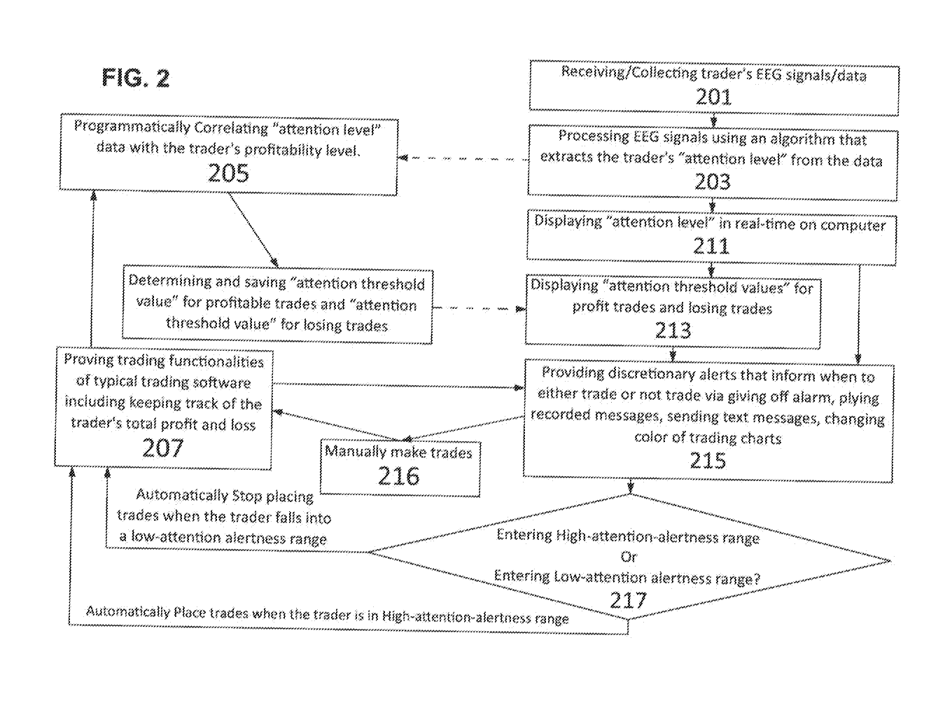

Referring to FIG. 2, there is disclosed the method in accordance with the principles of the present invention. The method includes software which providing features for both trading and analyzing EEG data. The trading feature includes functionalities of typical trading software and is known in the field and thus is not discussed herein.

After the trader's EEG data is received at step 201, the raw EEG data is processed using an algorithm that extracts the trader's attention level from the data at step 203. There are several algorithms available for extracting attention level from raw EEG data. Some algorithms are proprietary while other algorithms are in public domain. For example, a publication entitled “Learning EEG-based Spectral-Spatial Patterns for Attention Level Measurement” published in IEEE 2009 is a representative. (Brahim Hamadicharef, et. al., IEEE, 2009, http://www1.i2r.a-star.edu.sg/˜kptee/ISCAS09-attention.pdf.)

Most attention algorithms compare the changes between alpha, beta and theta brainwaves over time to compute the alertness level, while other algorithms also use delta and gamma brainwaves. The exact algorithm used is subjective so long as the “attention” output of the algorithm is a value that oscillates between two fixed values such as 1 and 100, or −50 and +50 for example. The attention data may be used in a raw form depending on the algorithm, or it may be smoothed using a moving average.

The software further has a training mode to help the trader to set up attention threshold values for profitable trades and losing trades before the trader applies the system/method in real-time trading.

When in “training mode”, the last t-seconds (t being between 1 to several seconds or minutes) of oscillating “attention level” data is programmatically correlated with the trader's profitability level at step 205. As the trader inputs “buy” and “sell” orders into the trading software, the software keeps track of the trader's total profit and loss while correlating the profitability with attention level at step 207. When it is found that the trader remains consistently profitable above a certain attention level, the attention threshold value is saved in the software at step 209 and applied to live trading at a later time. Likewise, the software also correlates lower attention levels with losing trades and saves the threshold value at step 209 to be later applied to live trading.

After the trader completes the “training mode”, the software should have learned the trader's alertness and profitability characteristics and may use those threshold values to alert the trader when to trade or not trade in live trading.

Nevertheless, the training mode can be bypassed if the trader chooses to.

In real-time trading, the software continuously processes the EEG data, programmatically learning patterns that are associated or correlated with winning or losing trades and updating the attention threshold values for winning trades and losing trades. The software will also display the attention level or alertness level on the computer screen at step 211. Optionally, the software can display the “attention threshold values” for profitable trades and losing trades on the computer at step 213. By visually representing the “alertness” or “attention” level on the computer screen as a graph so that the trader can actually see his or her own alertness level move in real-time, the trader is encouraged to attempt to keep his or her alertness' level up into the “profitable” range by concentrating. This form of biofeedback teaches the trader how to maintain a consistently elevated attention level that may possibly lead to more profitable trades or fewer losses.

The trading software may optionally play background meditation music of the trader's choosing to help maintain concentration levels.

At step 215, when the trader enters into a high-attention or low-attention alertness level, the software may sound an alarm or play a recording, such as a voice that says “Trade” or “Don't Trade”, “Stop” or “Go”, or the software may display a text message or picture alerting the trader to either trade or not to trade, or the software may color or modify the financial chart to represent the same. The trader is giving options to make trades at step 216 after thorough consideration of the system's alert. Optionally, alerts or alarms could be replaced with simply disabling trading functionality, automatically (programmatically) entering or exiting a trade.

The software may optionally perform fully automated trading based on real-time analysis of the trader's brainwave patterns at step 217. In this mode, the software will place trades automatically without any intervention from the trader. In conjunction with pre-defined trading rules either automatically generated by the software or defined by the trader, the software will place trades only when the trader is in a high-attention alertness level and will exit trades, reverse trades or stop placing trades when the trader falls into a low-attention alertness level of brainwave patterns.

It is known that EEG electrodes often lose signal strength and become unreliable when worn for an extended period of time or when moved out of place. When this occurs, the electrodes must be re-seated, cleaned, or gel must be added. For this reason it is possible that some discretionary alert signals may be the result of a bad EEG electrode connection. The software can display the EEG headset's signal strength so that when an alert is presented, the trader can reference the signal strength of the headset to determine if the alert signal is actually valid. The software algorithm may also take into account the EEG signal strength prior to issuing a discretionary alert.

Instead of using an “alertness” algorithm, raw EEG data could be used. Visual representation of a graph of alertness data isn't required. Alerts or alarms could be replaced with simply disabling trading functionality, automatically (programmatically) entering or exiting a trade.

The use of the system and method of the present invention is straightforward. A trader would install the software on his or her computer, install the drivers for the EEG device, plug the EEG device into the computer (either wired, wirelessly, or with Bluetooth), put the headset on the head, start the software and begin using it.

Whereas for purposes of simplicity of explanation, the methodologies are shown and described as a series of blocks, it is to be understood and appreciated that the claimed subject matter is not limited by the order of the blocks, as some blocks may occur in different orders and/or concurrently with other blocks from what is depicted and described herein. Moreover, not all illustrated blocks may be required to implement the methodologies described hereinafter.

While there have been shown and described and pointed out the fundamental novel features of the invention as applied to the preferred embodiments, it will be understood that the foregoing is considered as illustrative only of the principles of the invention and not intended to be exhaustive or to limit the invention to the precise forms disclosed. Obvious modifications or variations are possible in light of the above teachings. The embodiments discussed were chosen and described to provide the best illustration of the principles of the invention and its practical application to enable one of ordinary skill in the art to utilize the invention in various embodiments and with various modifications as are suited to the particular use contemplated All such modifications and variations are within the scope of the invention as determined by the appended claims when interpreted in accordance with the breadth to which they are entitled.

What is claimed is:

1. A method of analyzing EEG brain wave data in real time throughout the day and correlating the data with the attention level of a person transmitting the EEG signals while he/she is working at a job.

2. The method of claim 1, wherein the person is a day trader at any market in the world.

3. The method of claim 2, wherein the method comprising:
receiving the trader's EEG signals from headset to a computer that is running trading software;
processing EEG signals using algorithms that extract the trader's attention level from the data;
programmatically correlating attention level data with the trader's profitability level;
determining and saving attention threshold values; and
providing alerts that inform when to either trade or not trade.

4. The method of claim 3, the method further comprising automated trading wherein the software automatically places trades and stops placing trades based on the alertness level of brainwave patterns in conjunction with pre-defined trading rules either automatically generated by the software or defined by the trader.

5. The method of claim 3, wherein alerts provided by the software includes giving off an alarm or playing a recording of trading instruction or displaying text messages or pictures or changing colors of a financial chart to alert the trader to either trade or not to trade.

6. The method of claim 3, further comprising displaying a graph of alertness or attention level on the computer such that the trader can actually see his or her own alertness level move in real-time.

7. The method of claim 3, further comprising playing background meditation music of the trader's choice to help maintain concentration levels.

8. A system for analyzing EEG brain wave data in real time throughout the day and correlating the data with the attention level of the person transmitting the EEG signals while he/she is working at a job, wherein the system comprising:
an EEG device,
software which correlates the EEG data with the alertness or attention level of the person generating the EEG data; and
any type of computer capable of running the software and compatible with EEG device.

9. The system of claim 8, wherein the person is a day trader at any market in the world.

10. The system of claim 9, wherein the software further correlates the attention level data with the day trader's profitability level, determines and saves attention threshold values, and provides alerts that inform when to either trade or not trade.

11. The method of claim 10, wherein the software further provides automated trading wherein the software automatically places trades and stops placing trades based on the alertness level of brainwave patterns in conjunction with pre-defined trading rules either automatically generated by the software or defined by the trader.

12. The method of claim 10, wherein alerts provided by the software includes giving off an alarm or playing a recording of trading instruction or displaying text messages or pictures or changing colors of a financial chart to alert the trader to either trade or not to trade.

13. The method of claim 10, wherein the software further displays alertness or attention level on the computer such that the trader can actually see his or her own alertness level move in real-time.

14. The method of claim 10, wherein the software further plays background meditation music of the trader's choice to help maintain concentration levels.

15. A method of analyzing EEG brain wave data in real time throughout the day and correlating the data with trading patterns of a day trader transmitting the EEG signals, the method comprising:
receiving the trader's EEG signals from headset to a computer that is running trading software;
programmatically correlating the EEG signals with the trader's profitability level, the EEG signals being in a form of EEG signals raw data or attention level data extracted from the EEG signals;
Determining and saving attention threshold values; and
providing alerts that inform when to either trade or not trade.

16. The method of claim 15, the method further comprising automated trading wherein the software automatically places trades and stops placing trades based on the alertness level of brainwave patterns in conjunction with pre-defined trading rules either automatically generated by the software or defined by the trader.

17. The method of claim 15, wherein alerts provided by the software includes giving off an alarm or playing a recording of trading instruction or displaying text messages or pictures or changing colors of a financial chart to alert the trader to either trade or not to trade.

18. The method of claim 15, further comprising displaying alertness or attention level on the computer such that the trader can actually see his or her own alertness level move in real-time.

19. The method of claim 15, further comprising playing background meditation music of the trader's choice to help maintain concentration levels.

20. The method of claim 18, wherein the alertness or attention level is displayed in one of a variety forms including a graph, table, and any combinations thereof.
